(19) United States
(12) Patent Application Publication
(10) Pub. No.: US 2011/0112535 A1
(43) Pub. Date: May 12, 2011

(54) IM rod extension

(76) Inventor: Howard Martin Pecker, Westfield, NJ (US)

(21) Appl. No.: 12/925,982

(22) Filed: Jan. 12, 2011

Related U.S. Application Data
(60) Provisional application No. 61/280,605, filed on Nov. 6, 2009.

(51) Int. Cl. A61B 17/58

(52) U.S. Cl. 606/62

(57) ABSTRACT

The IM Rod extension is a metallic device, similar in size and shape to the proximal end of an IM Rod, meant to be attached to the distal end of an existing IM rod by distal exposure, and its purpose is to allow enhanced distal fixation of fractures, usually in the distal femur, by having variable sizes and greater option for direction and size of distal interlocking screw placement, and by being integrated with the existing IM Rod which maintains length and continuity with the rest of the femur.

### CROSS-REFERENCE TO RELATED APPLICATIONS

A Provisional patent for “IM Rod Extension” application Ser. No. 12/925,982 filing date Nov. 3, 2010 was filed by myself. There are no non-provisional patents that are related.

### STATEMENT REGARDING FEDERALLY SPONSORED RESEARCH (IF APPLICABLE)

No federal grant money was used for this patent.

### BACKGROUND OF THE INVENTION

IM rods are devices that consist of metal rods that are surgically implanted by orthopedic surgeons into fractured bones to stabilize the bone while it heals. IM rods that are currently in use have a wide proximal end, and a narrow distal end to facilitate their passing down the medullary cavity of a long bone. This design prevents the use of angled and multiple distal locking screws. Current rods may allow 2 to 3 distal locking screws, all of which must be placed at right angles to the rod, limiting the types of fractures that can be treated with this existing technique.

### SUMMARY OF THE INVENTION

The IM rod extension is a metallic structural extension that extends the length and girth of an existing IM rod and allows for the placement of more advantageously placed interlocking screws at the distal end of the rod, and greater numbers of screws for more secure and versatile fixation of fractures, mostly used at the distal femur, but not limited to this area. The retrograde placement of my invention, the IM Rod Extension, allows this larger diameter rod to be added to the end of an existing rod and, using the existing rods interlocking screws to fasten the extension in place, then use the extension with a rigid outrigger guide to place screws at more advantageous positions, and more distally in the bone, quickly and easily, and with less surgery required than to separately plate the bone, which is the only alternative. A specially mated IM rod can be used to maximize the effectiveness, and safety, of this technique, and these specially compatible rods made with existing IM rod technology, but with compatible distal threads and connection configurations are part of this claim.

### DESCRIPTION OF THE DRAWINGS

Two pages of drawings are included with a total of 4 figures.

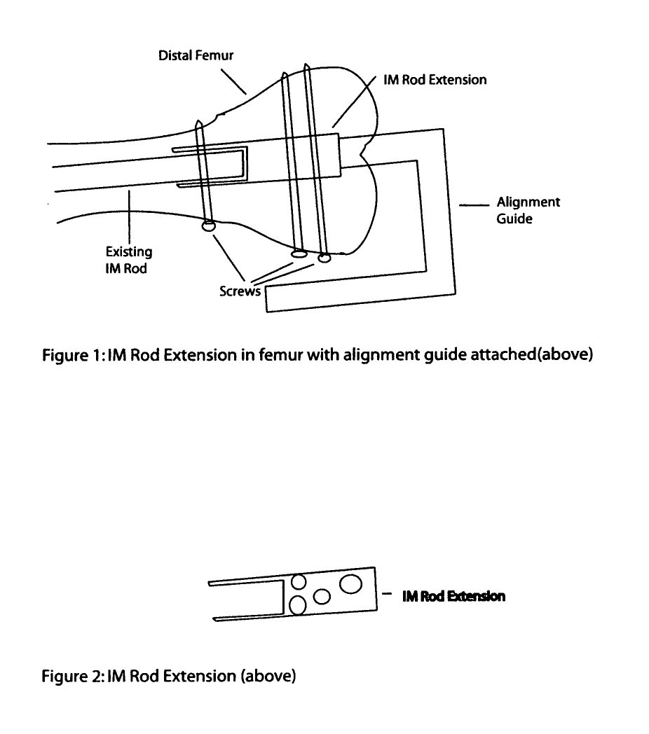

Page 1 of 2, FIG. 1: Shows a drawing of a distal femur with an IM rod, interlocking screws and an IM Rod Extension and an alignment guide.

Page 1 of 2, FIG. 2: Shows a drawing of the IM Rod Extension.

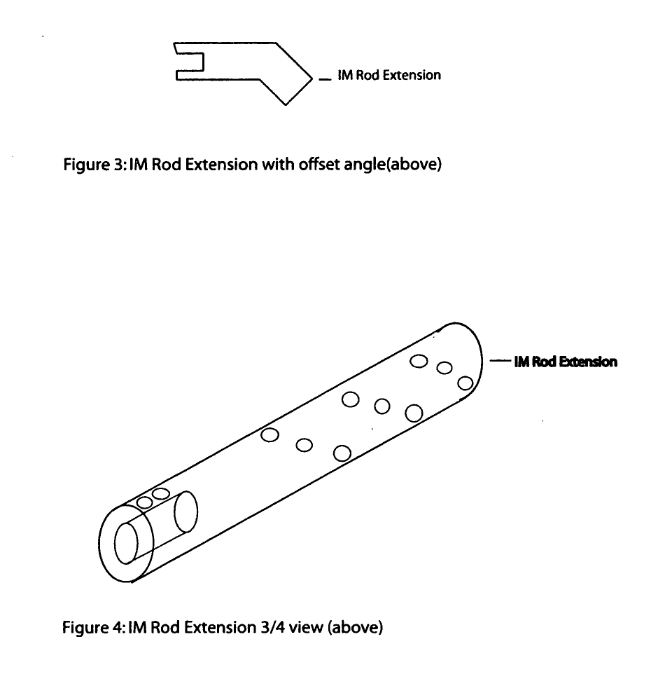

Page 2 of 2, FIG. 3: Shows an IM Rod Extension with angled shaft to accommodate ‘off’ rods.

Page 2 of 2, FIG. 4: Shows an perspective view of the IM Rod Extension.

### DETAILED DESCRIPTION OF THE INVENTION

The IM rod extension is a metallic structural extension that extends the length and girth of an existing IM rod and allows for the placement of more advantageously placed interlocking screws at the distal end of the rod, and greater numbers of screws for more secure and versatile fixation of fractures, mostly used at the distal femur, but not limited to this area. The retrograde placement of my invention, the IM Rod Extension, allows this larger diameter rod to be added to the end of an existing rod and, using the existing rods interlocking screws to fasten the extension in place, then use the extension with a rigid outrigger guide to place screws at more advantageous positions, and more distally in the bone, quickly and easily, and with less surgery required than to separately plate the bone, which is the only alternative. A specially mated IM rod can be used to maximize the effectiveness, and safety, of this technique, and these specially compatible rods made with existing IM rod technology, but with compatible distal threads and connection configurations are part of this claim. IM rods are devices that consist of metal rods that are surgically implanted by orthopedic surgeons into fractured bones to stabilize the bone while it heals. IM rods that are currently in use have a wide proximal end, and a narrow distal end to facilitate their passing down the medullary cavity of a long bone. This design prevents the use of angled and multiple distal locking screws. Current rods may allow 2 to 3 distal locking screws, all of which must be placed at right angles to the rod, limiting the types of fractures that can be treated with this existing technique.

### Description of Use

The IM Rod Extension is to be implanted by limited or full arthrotomy of the knee using a medial parapatellar incision common to that used with existing retrograde rods. It has an alignment guide similar to those currently in use with many other IM rod devices that utilize interlocking screws, except that it is designed to mate specially with the IM Rod extension by matching its size and length. Outriggers for the alignment guide will allow placement of interlocking screws at multiple angles to allow for fixation of complex comminuted fractures. This can be achieved with direct or indirect reduction methods. After initial exposure a drill hole is made just anterior to the notch of the femur to admit a flexible guide rod. This is inserted through the cancellus bone into the tip of the existing IM rod by x-ray guidance or direct vision. A flexible reamer enlarges the hole and a rigid guide is inserted into the rod. The offset from the center of the reamed opening to the rigid guide wire determines the offset selection of the available multi-angle IM Rod Extensions which is then inserted, with its alignment guide, over the end of the IM rod either by fluoroscopic guidance or direct vision, and held in place by either axial interlocking, if used with a compatible rod, or transverse interlocking screws.

I claim that my invention is the

1. IM rod extension,
which is an extension of an existing intramedullary (i.e. “IM”) rod of any type. It is an extra piece that fits over the end of an existing IM rod in order to allow improved fixation of a distal fracture fragment or impending fracture. It allows this by being larger in size than the existing rod, thereby allowing extra holes at extra angles (to accept interlocking screws) that are not currently available in a conventional IM rod. The extension is mated to an existing IM rod with a compatible distal end to accept the IM rod extension, or a universal extension that can fit over any suitable IM rod, determined by its composition and size, or over an IM rod specifically manufactured using current techniques to allow maximum compatibility with the IM Rod Extension. This claim covers all supportive hardware, such as alignments guides and compatible screw drivers, and IM Rod extensions of various configurations to accommodate different bones sizes and numbers and sizes of interlocking screws, and specifically manufactured compatible IM rods.
